(19) United States
(12) Patent Application Publication
(10) Pub. No.: US 2019/0239870 A1
(43) Pub. Date: Aug. 8, 2019

(54) LAPROSCOPIC KNOT APPLICATOR

(71) Applicant: Mahendra Thilanka Ratnapala, Delgoda (LK)

(72) Inventor: Mahendra Thilanka Ratnapala, Delgoda (LK)

(21) Appl. No.: 16/341,057

(22) PCT Filed: Mar. 23, 2017
(86) PCT No.: PCT/IB2017/005174

§ 371 Date: Apr. 10, 2019

(30) Foreign Application Priority Data
Oct. 24, 2016 (LK) 19048

(51) Int. Cl. A61B 17/04; A61B 17/29

(52) U.S. Cl. CPC A61B 17/0469; A61B 2017/291; A61B 2017/2906; A61B 17/29

(57) ABSTRACT

Laparoscopic Knot Applicator is a tool designed to facilitate application of “surgeons Knot” (FIG. 2) to ligate vessels and structures during laparoscopic procedures. Applying the secure surgeons knot in any surgical procedure is a life saving basic skill but it is a difficult task in laparoscopic surgeries. Operation of this tool (FIG. 3) ensures application of safer and secure knots compared to currently available techniques like clips, loops and Harmonic scalpel. Simple design of this tools enables laparoscopic surgeon to apply very secure conventional “surgeons knot” easily during laparoscopic surgeries even in a very limited surgical space. Cleaning and the sterilization of the instrument which is essential in surgical procedures is very easy as there are no electronic parts incorporated.

### TECHNICAL FIELD

Invention and the method described here is related to the procedure of ligating knots in Laparoscopic surgeries.

### BACKGROUND ART

Laparoscopy is a surgical technique involving surgeries of body cavities using keyhole incisions to insert elongated laparoscopic tools. In recent years it has replaced significant number of conventional open surgeries mainly due to its advantages to the patient such as less tissue damage, smaller scars, less pain and minimal hospital stay etc. Despite these advantages to the patient, it remains a big challenge to the laparoscopic surgeon to perform fine surgeries using long laparoscopic tools while observing on a screen. Surgeons need higher level of training and skills to perform laparoscopic surgeries efficiently with less complications like bleeding and damaging structures.

Newer instruments and methods are being introduced to minimize these limitations but never comparable to the sensitivity and efficiency of human hand which plays a big role in conventional open surgeries. During any surgical procedure surgeons may need tissue dissection, cutting structures and vessels, probing, ligation and anastomosing etc. For ligating structures surgeons prefer to use “surgeons knot” [FIG. 1] due to its very reliable knotting properties to occlude vessels and vital structures. In laparoscopic surgeries applying this secure surgeons knot is one of the most difficult and complex maneuver to the surgeon Therefore several alternative methods and instruments introduced to substitute ‘surgeons knot’ during past years. But secure ‘surgeons knot’ remains the method of choice for most surgeons, due to limitations and complications of alternative techniques.

Due to the significance of “surgeons Knot” in any type of surgery, there are several prior arts describing difference techniques of laparoscopic knot tying.

One category imitates the function of human hand which involves making throws (passing one end of the thread around the other end) to form a knot. This technique is used by surgeons to apply surgeons knot in open surgeries where hand can easily perform the operation effectively.

One of the prior arts following the above technique is—‘laparoscopic knot pushing device’ invented by Richard S. wilkenfeld enables surgeon to get both ends of a tying suture outside of the body cavity and apply the knot outside as in open surgery. Then the throws are pushed in side using knot pushing device to tighten the knot. This device use a completely different technique compared to the current invention which is capable of applying knots inside the body. When using this knot pushing device it is necessary several insertions and withdrawal of the device to apply a knot making it less efficient and time consuming. Another limitation of the knot pushing device is, the correct tension of applied knot is difficult to assess.

Another prior art based on making throws invented by Peter Fan, the Laparoscopic Dual grasper designed to perform surgeons knot inside the body cavity. This dual grasper is capable of making throws by holding and releasing each end of thread which resembles the function of hand knot tying. But as only two grasping jaws are in action it is obviously very less efficient than hand and may consume lot of time. Dual grasper needs lot of coordination to hold and release the thread time to time to make a throw of a knot.

Current invention does not use the principle of making throws as described above, but it is based on loop formation technique to apply knots.

George S. Ferzil's laparoscopic instrument is a multi functional tool which is using the loop formation technique for knot tying. Principle of applying knots in this method is based on formation of a loop on a one part of a thread and the other end is taken through this loop to form a knot.

Ferzil's instrument compared to the current invention does not hold the thread while making the loop. Instead it drapes the suture over the back side of the opened intermediate jaw which makes the thread highly unstable during instrument rotation to form a loop. Compared to the Ferzil's instrument the new instrument holds the thread within the thread holding groove ensures stable rotation of thread during loop formation. Thread holding groove of the current invention has the ability of catching the thread throughout its circumference which is very convenient to the surgeon

In the process of making loops Ferzil's device needs to rotate the whole instrument including handles, but the new invention's rotation function is very effective due to the action of rotation controller which only rotates shaft while stabilizing the handle in any convenient position to the operating surgeon. For ligation shorter threads can be used in current invention as the thread holding groove catching the thread from end points compared to Ferzil's instruments which needs longer threads to drape from a middle point making it less economical.

The closest prior art described here the Ferzils laparoscopic instrument is completely different from the current invention in several aspects. The new invention described here has a thread holding groove which is having a capacity to hold the thread around its whole circumference. There fore to catch the thread new instrument does not need to turn to the direction of the thread as in Ferzils instrument.

Most significant novelty of the current invention is that, the rotation of the thread in loop formation process can be achieved by rotating the shaft of the instrument using the rotation controller only[without rotating the handle of the instrument]. This feature of the current invention significantly improve the efficiency of the knot ligation procedure never archived by any other prior art as they were designed with two separate jaws where rotation of the shaft/grasping jaws independent to the handle was mechanically impossible.

### SUMMARY OF THE INVENTION

Ligation of knots in a very limited space inside the body cavity, using long instruments having only two jaws is always a great challenge. Followings are the currently available alternatives and their limitations associated.

Clips—made out of metal or plastic is the widely used alternative to ligation. Clips are applied using a Clip-Applicator and only suitable for smaller diameter (<7 mm) vessels and ducts. If higher clamping pressures are applied on tissues clip may cause tissue necrosis and perforation. It is not suitable for inflamed structures like appendix as it may cut through inflamed tissues. Risk of clips slipping away is significantly higher than conventional surgical knots.

Loops—preformed loop of thread using surgical suture materials. This technique applicable only to structures with a free end like appendix. In laparoscopic appendix removal surgeries, appendix is trapped into the preformed loop and fastening of the knot is done by pulling the string out. Risk of easy unfastening and loop is not applicable to blood vessels (as there is no free end) are the main limitations.

Harmonic scalpel—most advanced technology using ultrasound waves to ablate and cut tissues and blood vessels. This technique also not applicable to larger blood vessels. It only coagulates blood vessels but does not ensure perfect sealing effect always compared to ligation. This technology is very expensive and usually not affordable to underdeveloped countries

As a solution to the above mentioned limitations and to facilitate the surgeon preferred long practiced conventional ligation technique, this new instrument and the method developed.

Main advantages of this invention are as follows;

I. Ligation procedure needs minimal effort and skills compared to the current laparoscopic ligation maneuver.
ii. Risk of slip ligature and cut through is minimal
iii. This technique is applicable to larger structures where other methods may failed.
iv. For ligations using this instrument, standard surgical suture materials are used, therefore the cost is very low.
v. this technique is very effective even with very narrow instrument angles where ligations using standard method may be very difficult.

### BRIEF DESCRIPTION OF DRAWINGS

Diagrams and the illustrations shown here will help to understand the structure of the preferred embodiment of the invention and its functional capabilities.

FIG. 1. conventional “surgeons knot” basic configuration.

FIG. 2. laparoscopic knot applicator structure and components.

FIG. 3. step by step illustration showing ligation procedure using laparoscopic knot applicator.

### DESCRIPTION OF EMBODIMENTS

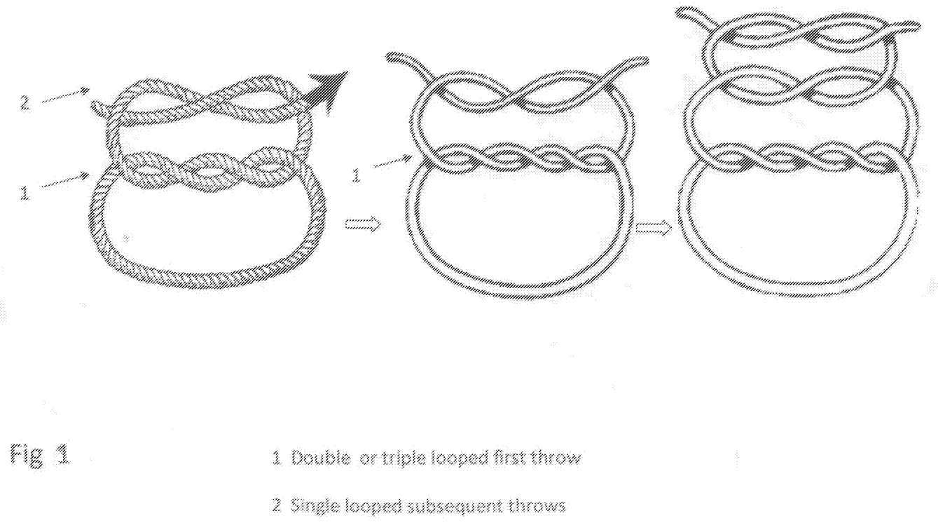

Laparoscopic knot applicator is a tool designed to facilitate easy application of surgeons knot in laparoscopic surgeries. Before going in to detailed description of the instrument it is essential to have an idea of basic configuration of “surgeons knot” [FIG. 1] and its unique knotting properties.

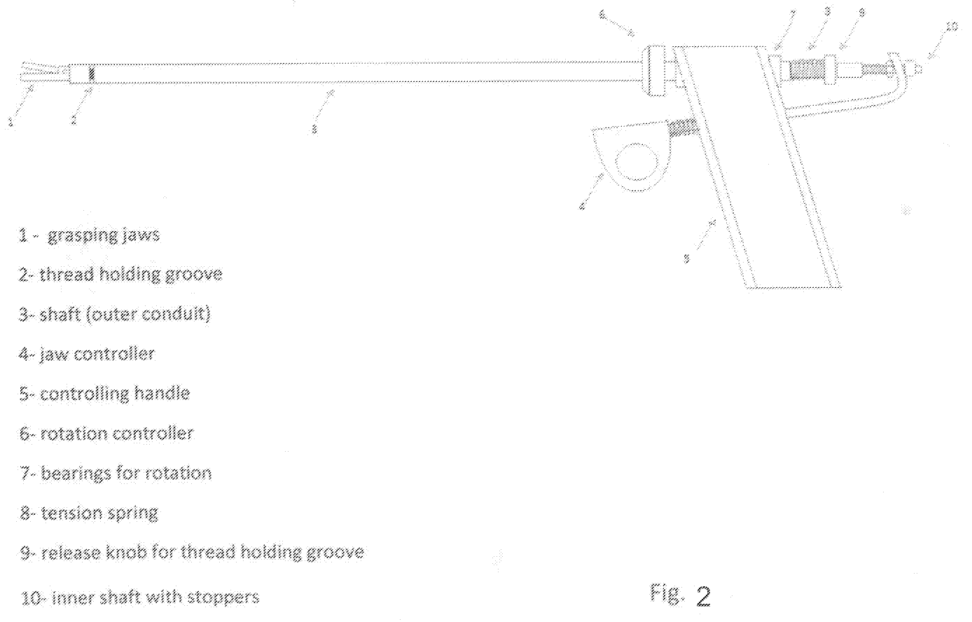

During any kind of surgical practice application of secure “surgeons knot” plays a very vital life saving role. Surgeons knot is a type of modified “reef knot”. As illustrated in FIG. 1, typical surgeons knot has double loops (twists) in its first throw[FIG. 1-1] and two subsequent throws having only one loop[FIG. 1-2]. The significance of this configuration is, double looped first throw ensures good friction and tethering to the ligated tissue which prevents slip ligature. Double twisted first throw also helps to maintain the tension of the first throw while applying the subsequent knots. Second and third throws of the knot prevents unfastening of the knot due to tension, pressure inside vessels etc. Due to this reliable and tissue friendly characteristics of this knot surgeons preferred to use it where ever necessary.

In contrast to open surgeries, it is not an easy task to perform the knot in laparoscopic procedures as surgeons have to use long instruments within a limited space. When laparoscopic ligating instruments parallel to each other (or narrow manipulation angle is used) it is extremely difficult to negotiate the loops of the knot. The new instrument described here helps to overcome these problems easily.

As illustrated in FIG. 1.1 double twist of first throw needs rotation of 720 degrees rotation of one thread around the other. performing these rotations inside body using long instruments, while watching on a two dimensional monitor in a small space is extremely difficult and needs lot of practice and coordination. Invention described here significantly reduce the effort and time necessary to apply knots during Laparoscopy.

preferred embodiment of the invention as shown in FIG. 2. comprised of a pair of grasping jaws[1], thread holding groove[2], shaft of the tool[3], jaw controller[4], controlling handle[5], rotation controller[6], bearings for rotation[7], tension springs[8], release knob for thread holding groove[9] and a inner shaft with stoppers.

Basic structure of the instrument can be described as a standard laparoscopic needle holder (or a grasper) with improvements comprising of a thread holding groove[2] and a release knob for the thread holder[9].

Grasping jaws[1] can be opened and closed using the jaw controller[4] to catch tissues and thread. whole shaft[3] of the instrument including grasping jaws[1] and thread holding groove[2] is rotatable in either direction using the rotation controller knob[6]. Thread holding groove[2] is kept closed position using tension spring[8] and opened only by pressing the release knob [9]. During ligation procedure once the thread is fed to the thread holding groove[2] it retains inside the groove[2] till released by the release knob[9]. This self retaining property of thread holding groove[2] is essential for the proper functioning of the knot applicator.

As illustrated on drawings present invention is entirely a mechanical device which contains no electronics or batteries. This is an important property of reusable surgical tools as these instruments need regular cleaning and sterilization using liquid disinfectants etc.

### Operation of the Instrument.

Laparoscopy is a surgical technique developed to perform operations in side the body cavity using keyhole incisions. Carbon dioxide gas is pumped in to the abdominal cavity of anesthetized patient to achieve operating space. Then laparoscopic camera is inserted through a small incision at umbilicus to visualize intra abdominal structures on a TV screen. Instruments necessaryto perform operation are inserted through several other ports created on abdominal wall.

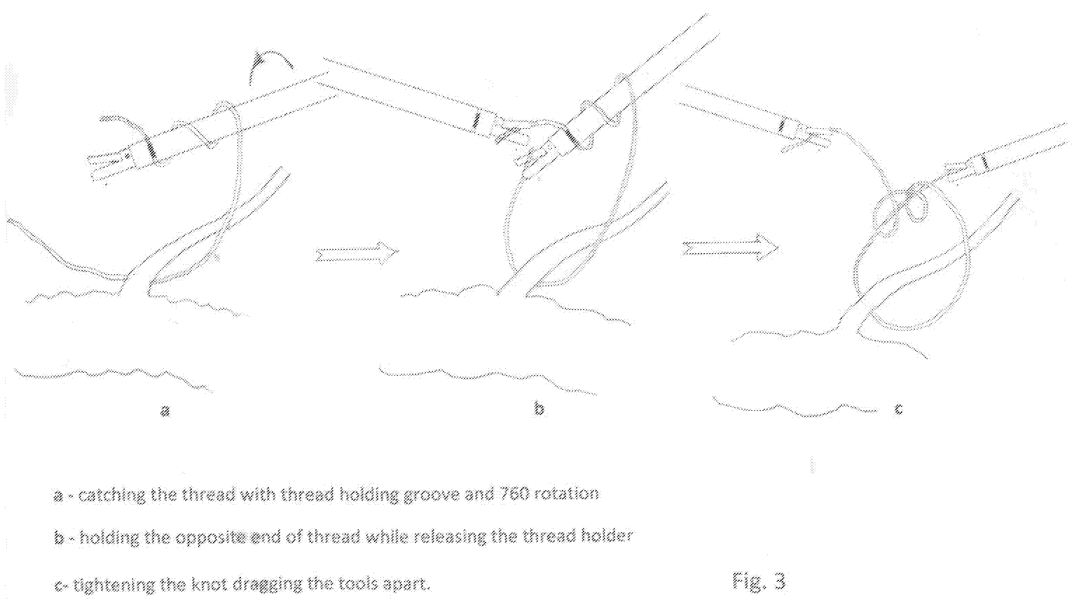

Invention described here is a laparoscopic tool to perform litigation in side the abdominal cavity. Once the vessel or the structure to be ligated is defined, suitable type of surgical suture material is inserted in to the body cavity via one of the ports. Then the thread is placed behind the vessel and one free end of thread is fed to the thread holding groove[FIG. 1-2] of the knot applicator after opening the groove by pressing thread release knob[FIG. 1-9]. Once caught by the thread holding groove thread will retain there due to the action of the tension spring. Next as illustrated on FIG. 3 thread is twisted around the shaft of the instrument 720 degrees using the rotation controller knob [FIG. 1-6] in clockwise direction. Now the other end of the thread is caught by the grasping jaws of the same instrument while the vessel/structure is encircled as demonstrated in FIG. 3-b. Now use a second instrument to catch the end of the thread held by the thread holding groove and drag it away to release the thread from the groove. While holding both ends of the string tightly, pull both instruments apart making the double twisted first throw of the ‘surgeons knot’ successfully. Subsequent throws of the knot can be performed easily repeating the same sequence as described above.

Current invention can be used in a different way to apply the first throw of the ‘surgeons knot’ as follows. Here the suture thread is fed to the thread holding groove and twist it around the shaft of the knot applicator 270 degrees, before inserting into the body cavity. Now the knot applicator with the twisted thread is introduced in to the abdominal cavity through a port. Then using a second grasping instrument pass the free end of the thread around the structure to be ligated and feed that end of the thread to the knot applicator grasping jaws. Next pull the end of the thread at the thread holding groove using the second instrument to release it. Now drag both ends of the thread apart to apply the first throw of the surgeons knot.

1. A device for making knots in laparoscopic surgery wherein the said device is comprising:
a. a pair of grasping jaws[1] to grasp and manipulate tissues and surgical thread,
b. a thread holding groove[2] placed proximal to the said grasping jaws assists in holding thread during loop formation of knot application,
c. a shaft [3] rotatable in either direction totally independent from the controlling handle [5] to which said grasping jaws [1] and the thread holding groove [2] are attached,
d. a jaw controller[4] at the controlling handle[5] operating said grasping jaws[1] open and close,
e. a controlling handle[5] to which the shaft[3] of the instrument is attached independently rotatable,
f. a rotation controller[6], connected to the shaft [3] at the handle end of the device assisting rotation in either direction,
g. bearings for rotation[7] connecting the said shaft[3] to the controlling handle,
h. tension springs[8] connected to the release knob for thread holding groove[9] to keep the said thread holding groove [2] at closed position,
i. a release knob for thread holding groove[9] permitting opening of the said thread holding groove[2] to load and release the thread.

2. The device claimed in claim 1 comprising a thread holding groove wherein the said thread holding grove [FIG. 1-2] is an adjustable groove placed proximal to the grasping jaws [FIG. 1-1] of the instrument and the thread holding groove [FIG. 1-2] is kept closed position normally and opened using the release knob[FIG. 1-9] to load and release the thread.

3. The device claimed in claim 1 comprising a thread holding groove where in the said holding groove [FIG. 1-2] is an adjustable gap around the whole circumference of the shaft of the tool [FIG. 1-3]

4. The device claimed in claim 1 comprising a thread holding groove wherein the said holding groove[FIG. 1-2] is any type of second grasping area or grasping jaws in addition to the main grasping jaws [FIG. 1-1].

5. The device claimed in claim 1 comprising a thread holding groove wherein the said holding grove [FIG. 1-2] is capable of rotating in either direction with the aid of the rotation controller [6] at the handle end without rotating the controlling handle [5].

6. A method for applying instrumental ‘surgeons knot’ [FIG. 2] comprising a rotatable thread holding groove[FIG. 1-2] which can hold and wrap the surgical thread around the independently rotatable shaft to any degree of rotation to form loops in the process of knot application.

7. A method of applying intracorporeal surgeons knot in laparoscopic procedure involving making a loop at one end of the thread where other end is passed through to form a knot.
Method described herein is performed by holding one end of the thread using said thread holding groove and wrapping it around the independently rotating shaft of the tool making necessary number of loops. Once the said loop is formed around the shaft, the free end of the same thread is tightly held by the grasping jaws while wrapped end is released and dragged apart using second tool making the first knot.
